(19) United States
(12) Patent Application Publication
(10) Pub. No.: US 2019/0167239 A1
(43) Pub. Date: Jun. 6, 2019

(54) SURGICAL-DEVICE ROTATION MECHANISM

(71) Applicant: OLYMPUS CORPORATION, Tokyo (JP)

(72) Inventors: Hiroshi OKABE, Tokyo (JP); Masahiro FUJII, Tokyo (JP); Masatoshi IIDA, Tokyo (JP); Yoshiyuki KUMADA, Tokyo (JP)

(73) Assignee: OLYMPUS CORPORATION, Tokyo (JP)

(21) Appl. No.: 16/266,167

(22) Filed: Feb. 4, 2019

Related U.S. Application Data
(63) Continuation of application No. PCT/JP2016/081281, filed on Oct. 21, 2016.

(51) Int. Cl. A61B 17/00

(52) U.S. Cl. CPC A61B 17/00; A61B 2017/2929; A61B 2017/00557

(57) ABSTRACT

A surgical-device rotation mechanism according to the present invention includes: a long member; a rotation member rotatably supported by a distal end of the long member and rotates around a longitudinal axis of the long member; an end effector that is fixed to the rotating member; a tension converting member disposed inside the long member along the longitudinal axis and has a distal end to which the rotation member is fixed, and converts tension into rotation around the longitudinal axis; and a rotation restriction mechanism disposed between the long member and the tension converting member and increases or decreases friction between the rotation restriction mechanism and at least one of an inner surface of the long member and an outer surface of the tension converting member so as to restrict or not restrict relative rotation between the long member and the tension converting member around the longitudinal axis.

### CROSS-REFERENCE TO RELATED APPLICATIONS

This is a continuation of International Application PCT/JP2016/081281, with an international filing date of Oct. 21, 2016, which is hereby incorporated by reference herein in its entirety.

### TECHNICAL FIELD

The present invention relates to surgical-device rotation mechanisms.

### BACKGROUND ART

With regard to a known surgical device that causes an end effector, which is disposed at the distal end of a long member formed of a coil sheath to be inserted into a biological organism, to operate by pulling of a wire, a coil-sheath winding direction and a wire twisting direction are matched so that, by utilizing untwisting of the wire in accordance with the pulling force, the end effector is rotated around the longitudinal axis of the long member (for example, see Japanese Unexamined Patent Application, Publication No. Sho 64-26017).

### SUMMARY OF INVENTION

An aspect of the present invention provides a surgical-device rotation mechanism including: a long member formed tubular shape; a rotation member that is rotatably supported by a distal end of the long member around a longitudinal axis of the long member; an end effector that is fixed to the rotating member; a tension convertion member that is disposed inside the long member along the longitudinal axis of the long member and has a distal end to which the rotation member is fixed; and a rotation restriction mechanism that is disposed between the long member and the tension convertion member. The tension convertion member is configurd to convert tension applied by pulling of a proximal end into rotation around the longitudinal axis at the distal end. The rotation restriction mechanism is configured to increase or decrease friction between the rotation restriction mechanism and at least one of an inner surface of the long member and an outer surface of the tension convertion member by being operated from a proximal end of the long member so as to restrict or not restrict relative rotation between the long member and the tension convertion member around the longitudinal axis.

### BRIEF DESCRIPTION OF DRAWINGS

FIG. 1 is a partial side view illustrating a surgical-device rotation mechanism according to an embodiment of the present invention.

FIG. 2 is a partial vertical sectional view illustrating the surgical-device rotation mechanism in FIG. 1.

FIG. 3 is a partial vertical sectional view illustrating a state where a twisted wire of the surgical-device rotation mechanism in FIG. 1 is pulled toward the proximal end.

FIG. 4 is a partial vertical sectional view illustrating a state where a rotation restriction mechanism is actuated in the surgical-device rotation mechanism in FIG. 3.

FIG. 5 is a partial vertical sectional view illustrating a first modification of the surgical-device rotation mechanism in FIG. 1.

FIG. 6 is a partial vertical sectional view illustrating a state where a rotation restriction mechanism is actuated in the surgical-device rotation mechanism in FIG. 5.

FIG. 7 is a partial vertical sectional view illustrating a second modification of the surgical-device rotation mechanism in FIG. 1.

FIG. 8 is a partial vertical sectional view illustrating a state where the twisted wire of the surgical-device rotation mechanism in FIG. 7 is pulled toward the proximal end.

FIG. 9 is a partial vertical sectional view illustrating a state where a rotation restriction mechanism is actuated in the surgical-device rotation mechanism in FIG. 8.

FIG. 10 is a perspective view illustrating an example of a mechanism that regulates relative movement between a first friction member and a coil sheath in the surgical-device rotation mechanism in FIG. 7.

FIG. 11 is a perspective view illustrating a modification of the surgical-device rotation mechanism in FIG. 7.

FIG. 12 is a perspective view illustrating another modification of the surgical-device rotation mechanism in FIG. 7.

FIG. 13 is a perspective view illustrating a modification of the mechanism that regulates relative movement between the first friction member and the coil sheath in FIG. 10.

### DESCRIPTION OF EMBODIMENTS

A surgical-device rotation mechanism 1 according to an embodiment of the present invention will be described below with reference to the drawings.

The surgical-device rotation mechanism 1 according to this embodiment causes an end effector (surgical device) 3, such as forceps, attached to the distal end of a flexible tubular coil sheath (long member) 2 to rotate around a longitudinal axis of the coil sheath 2 in response to an operation performed at the proximal end of the long member 2.

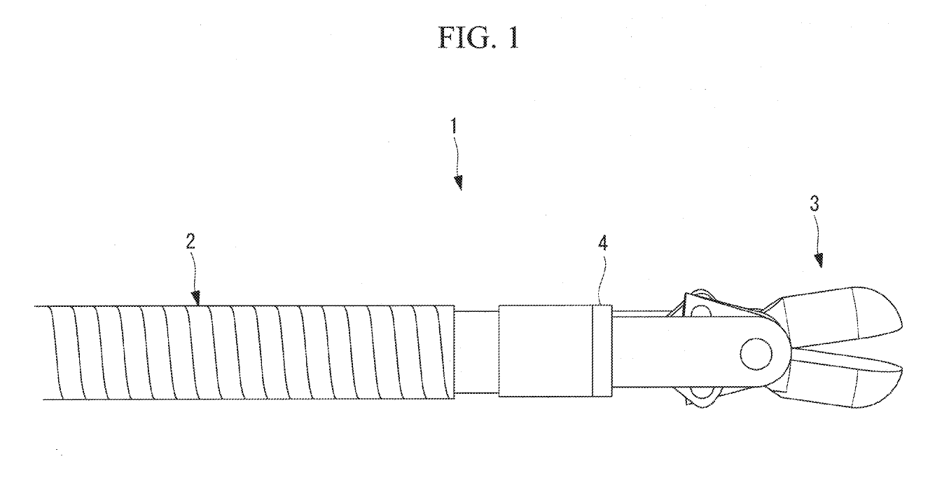

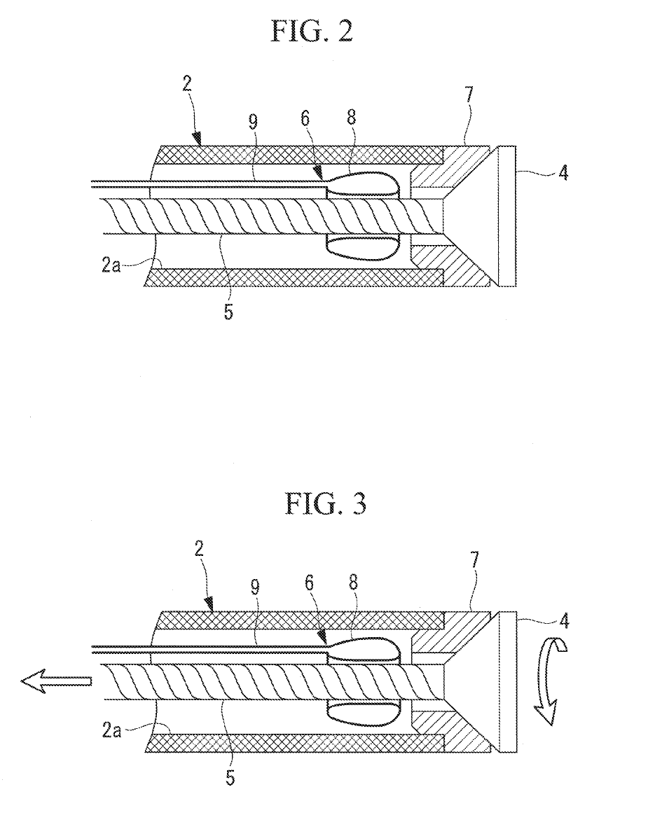

As shown in FIGS. 1 and 2, the surgical-device rotation mechanism 1 according to this embodiment includes a flexible tubular coil sheath 2, a cylindrical rotation member 4 supported in a rotatable manner about its longitudinal axis by the distal end of the coil sheath 2, a flexible tubular twisted wire (tension convertion member) 5 that extends through a center hole 2a of the coil sheath 2 and along the entire length of the coil sheath 2 and that has the rotation member 4 fixed to the distal end thereof, and a rotation restriction mechanism 6 disposed between the twisted wire 5 and the coil sheath 2.

The end effector 3 is fixed to the rotation member 4.

As shown in FIG. 2, a receiver member 7 with which the proximal-end surface of the rotation member 4 is brought into contact in the longitudinal direction and that supports the rotation member 4 in a rotatable manner about its longitudinal axis is fixed to the distal end of the coil sheath 2. The contact surfaces of the receiver member 7 and the rotation member 4 have reduced surface roughness so that the friction therebetween is as small as possible.

When a predetermined tension or larger is generated at the proximal end of the coil sheath 2 in accordance with a pulling force applied manually or from an actuator (not shown), the twisted wire 5 moves in the unraveling direction. This causes a rotational force acting in the untwisting direction around the longitudinal axis to be generated in the twisted wire 5. In other words, the twisted wire 5 converts the pulling force applied in the longitudinal direction to the proximal end of the twisted wire 5 into a rotational force around the longitudinal axis at the distal end of the twisted wire 5.

The rotational force converted from the tension by the twisted wire 5 causes the rotation member 4 fixed to the distal end of the twisted wire 5 to rotate around the longitudinal axis, thus causing the end effector 3 fixed to the rotation member 4 to rotate around the longitudinal axis.

As shown in FIGS. 2 to 4, the rotation restriction mechanism 6 includes a ring-shaped balloon (friction member) 8 disposed near the distal end of the coil sheath 2 and between the inner surface of the coil sheath 2 and the outer surface of the twisted wire 5, a pipe (operation device) 9 whose distal end is connected to the balloon 8 and whose proximal end extends outward from the proximal end of the coil sheath 2, and a pump 10 such as a syringe that supplies a fluid, such as liquid or gas, into the balloon 8 via the pipe 9.

Accordingly, the balloon 8 contracts when the fluid is discharged from the balloon 8 by the pump 10, whereas the balloon 8 expands and comes into close contact with the inner surface of the coil sheath 2 and the outer surface of the twisted wire 5 when the fluid is supplied into the balloon 8 by the pump 10.

The operation of the surgical-device rotation mechanism 1 according to this embodiment having the above-described configuration will be described below.

As shown in FIG. 2, the surgical-device rotation mechanism 1 according to this embodiment uses the pump 10 to discharge the fluid from the balloon 8 so as to cause the balloon 8 to contract. In this state, as shown in FIG. 3, the surgical-device rotation mechanism 1 applies a pulling force for pulling the twisted wire 5 toward the proximal end from the proximal end of the coil sheath 2. The applied pulling force generates tension in the twisted wire 5, and the generated tension is transmitted to the rotation member 4 at the distal end of the coil sheath 2, thus pulling the rotation member 4 toward the proximal end. Because the rotation member 4 is in contact with the receiver member 7 in the axial direction, the rotation member 4 does not move in the axial direction, and the tension generated in the twisted wire 5 increases.

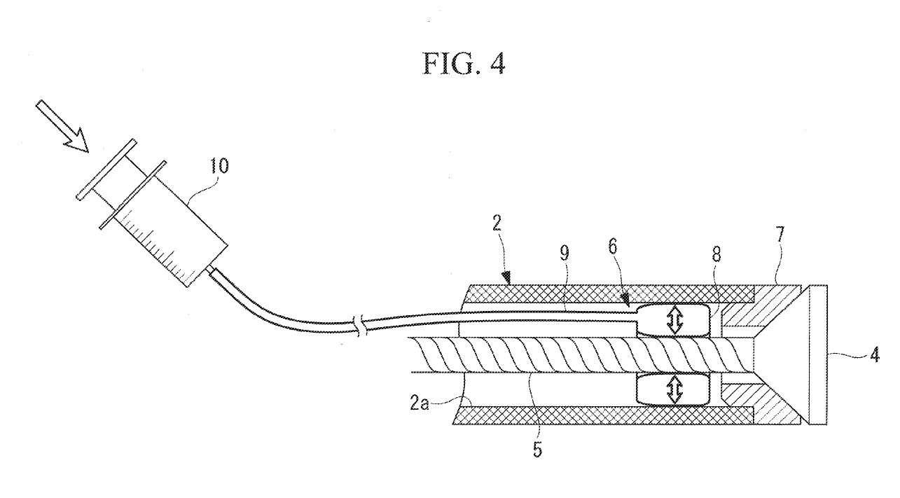

When the tension generated in the twisted wire 5 reaches a predetermined value or larger, the twisted wire 5 moves in the unraveling direction while being stretched by the tension, as shown in FIG. 4, thereby causing a rotational force in the untwisting direction to occur at the distal end of the twisted wire 5. Because the rotation member 4 fixed to the distal end of the twisted wire 5 is rotatably supported with small friction around the longitudinal axis by the receiver member 7 fixed to the distal end of the coil sheath 2, the rotation member 4 is rotated around the longitudinal axis, as indicated by an arrow, by the rotational force generated at the distal end of the twisted wire 5. Consequently, the end effector 3 fixed to the rotation member 4 is also rotated around the longitudinal axis.

When the twisted wire 5 is untwisted as a result of the rotation of the rotation member 4, the tension generated in the twisted wire 5 is released, thus causing the rotation member 4 to stop rotation at that position. If the end effector 3 is to be further rotated, a pulling force for pulling the proximal end of the twisted wire 5 toward the proximal end is applied again, so that the end effector 3 can be rotated again in the same direction around the longitudinal axis.

When the pulling force applied to the twisted wire 5 is reduced, the tension decreases accordingly, thus causing the twisted wire 5 to move in the untwisting direction. Thus, the rotation member 4 is rotated in the direction for untwisting the twisted wire 5, so that the rotation member 4 can be returned to its initial rotational angle position.

In this case, at an intermediate rotational angle position where the rotation member 4 is rotated relative to the receiver member 7 and the end effector 3 fixed to the rotation member 4 is rotated around the longitudinal axis of the coil sheath 2, the surgical-device rotation mechanism 1 according to this embodiment actuates the pump 10 so as to supply the fluid into the balloon 8, thereby causing the balloon 8 to expand, as shown in FIG. 4. The expanded balloon 8 comes into close contact with the outer surface of the twisted wire 5, at the inner side in the radial direction, and the inner surface of the coil sheath 2, at the outer side in the radial direction, so that the twisted wire 5 can be inhibited from rotating around the longitudinal axis relative to the coil sheath 2.

Specifically, with the surgical-device rotation mechanism 1 according to this embodiment, the rotation member 4 can be fixed at any rotational angle position even without continuously applying a pulling force to the twisted wire 5, so that an approach angle to a target site for the end effector 3 fixed to the rotation member 4 can be maintained. Consequently, the target site can be readily treated.

Accordingly, the surgical-device rotation mechanism 1 according to this embodiment is advantageous in that it can rotate the end effector 3 around the longitudinal axis of the coil sheath 2 and maintain the end effector 3 at any rotational angle position.

In this embodiment, the balloon 8 is not fixed to the inner surface of the coil sheath 2 or the outer surface of the twisted wire 5 and comes into close contact therewith by expanding. Alternatively, the balloon 8 may be fixed to either one of the two. By restricting sections where relative movement is inhibited by friction, such sections can be secured more reliably. In particular, it is preferable that the balloon 8 be fixed to the outer surface of the twisted wire 5 and that the coil sheath 2 and the balloon 8 be the sections to be secured by friction. Even if the frictional force is small, a larger torque can be generated.

As an alternative to this embodiment in which the rotation restriction mechanism 6 is constituted by the balloon 8, the pipe 9, and the pump 10, other configurations may be employed.

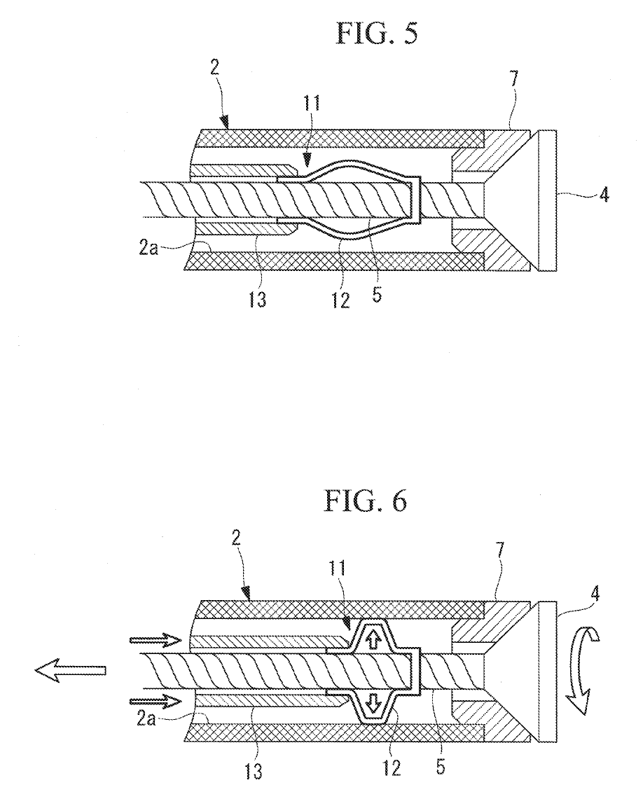

For example, in an example shown in FIGS. 5 and 6, a rotation restriction mechanism 11 includes strip-like plate spring members (expansion-contraction member, friction member) 12 each of which has its distal end fixed to an intermediate position of the twisted wire 5 in the longitudinal direction, and also includes a push-fit coil (power transmission member, operation device) 13 that is connected to the proximal ends of the plate spring members 12 and that supplies a pressing force for pressing the proximal ends of the plate spring members 12 toward the distal ends. The plate spring members 12 are provided at, for example, four locations spaced apart in the circumferential direction of the twisted wire 5, and the midsection of each member in the lengthwise direction is slightly bent radially outward. The number of plate spring members 12 is arbitrary.

The push-fit coil 13 has a tubular shape that allows the twisted wire 5 to extend therethrough. The proximal end of the push-fit coil 13 extends outward from the proximal end of the coil sheath 2, whereas the distal end of the push-fit coil 13 has the proximal ends of all of the plate spring members 12 fixed thereto.

By operating the push-fit coil 13 at the proximal end of the coil sheath 2 and pushing the push-fit coil 13 toward the distal end, a pressing force is applied to the proximal ends of all of the plate spring members 12. As shown in FIG. 6, the plate spring members 12 receiving the pressing force at the proximal ends thereof deform such that the bent midsections thereof are further bent, thus protruding radially outward. Consequently, the midsections of the plate spring members 12 bent with a large curvature are brought into close contact with the inner surface of the coil sheath 2 so that friction occurs between the two, whereby the twisted wire 5 is maintained such that it does not rotate around the longitudinal axis relative to the coil sheath 2.

When the pressing force applied to the push-fit coil 13 is released, the elastic restoring force of each plate spring member 12 causes it to return in the direction in which the curvature thereof decreases, so that the bent midsection moves away from the inner surface of the coil sheath 2, whereby the secured state is released.

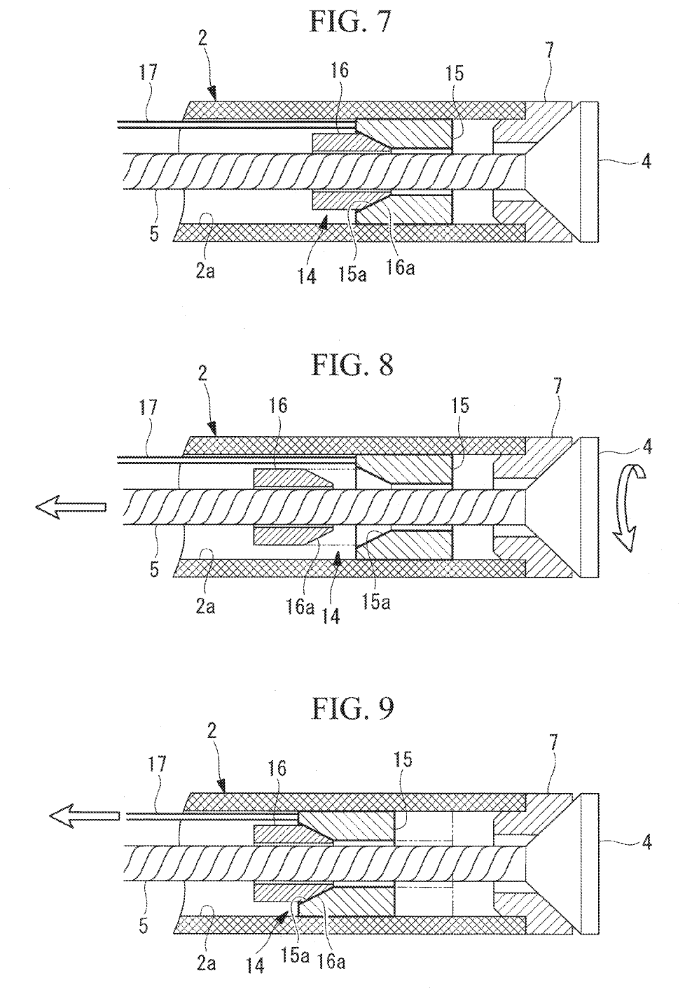

In an example shown in FIGS. 7 to 10, a rotation restriction mechanism 14 includes a first friction member 15 provided in the coil sheath 2, a second friction member 16 provided in the twisted wire 5, and a wire (power transmission member) 17 that moves the first friction member 15 in the axial direction.

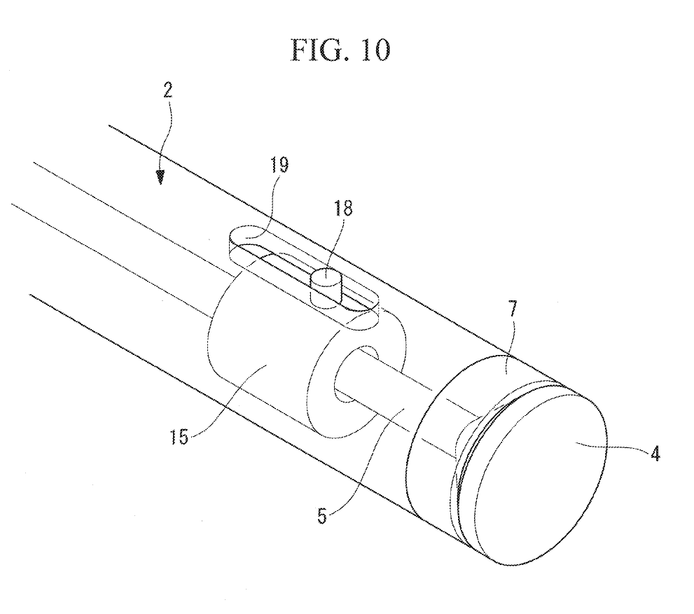

The first friction member 15 is a substantially cylindrical member disposed along the longitudinal axis of the coil sheath 2 and has a conically tapered inner surface 15a that tapers from the proximal end toward the distal end. As shown in FIG. 10, the first friction member 15 is movable along the longitudinal axis relative to the coil sheath 2 by accommodating a protrusion 18, which extends radially outward from the outer surface, in a slit 19 formed in the coil sheath 2 along the longitudinal axis, but is not relatively rotatable around the longitudinal axis.

The second friction member 16 is a substantially cylindrical member fixed along the longitudinal axis to the outer surface of the twisted wire 5 and includes a conically tapered surface 16a, which is complementary to the tapered inner surface 15a of the first friction member 15, at the distal end.

As shown in FIG. 8, when the twisted wire 5 is pulled toward the proximal end, the twisted wire 5 stretches and moves in the unraveling direction, so that the first friction member 15 and the second friction member 16 move away from each other along the longitudinal axis of the coil sheath 2, whereby the rotation member 4 and the end effector 3 fixed to the distal end of the twisted wire 5 can be rotated around the longitudinal axis of the coil sheath 2.

In this state, the wire 17 is pulled toward the proximal end from the proximal end of the coil sheath 2, so that the first friction member 15 is moved toward the proximal end along the longitudinal axis of the coil sheath 2, thereby bringing the tapered inner surface 15a into close contact with the tapered surface 16a of the second friction member 16, as shown in FIG. 9. Thus, a state where the tapered inner surface 15a and the tapered surface 16a are secured to each other can be achieved in accordance with friction generated therebetween. Consequently, even when the pulling force applied to the twisted wire 5 is reduced, a predetermined section is maintained at a certain rotational angle position around the longitudinal axis.

Because the conically tapered surface 16a of the second friction member 16 is in close contact with the conically tapered inner surface 15a of the first friction member 15, the twisted wire 5 and the coil sheath 2 can be secured to each other at any relative rotational angle position. In other words, this is advantageous in that the relative rotational angle position at which the components can be secured to each other can be changed continuously.

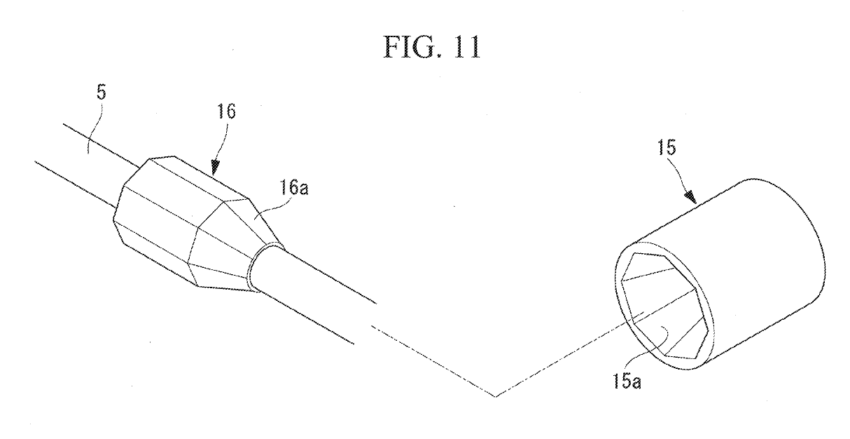

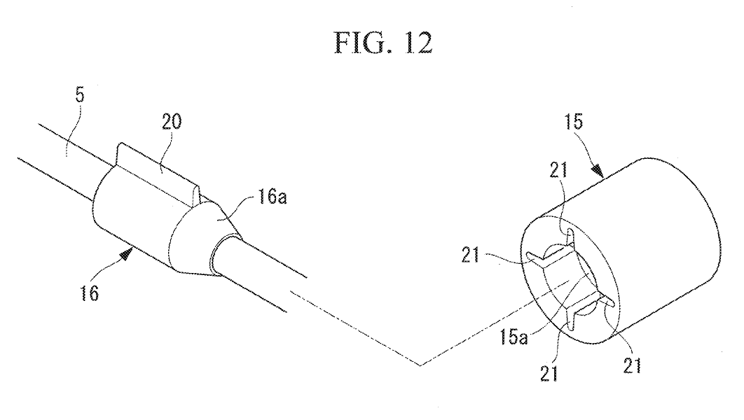

Alternatively, as shown in FIG. 11, the tapered inner surface 15a may be a pyramidal inner surface, and the tapered surface 16a may be a pyramidal surface complementary to the tapered inner surface 15a. Consequently, the relative rotational angle changes in a stepwise fashion when the tapered inner surface 15a and the tapered surface 16a are brought into close contact with each other, but the frictional force when the two surfaces are brought into close contact with each other can be increased, whereby the two surfaces can be secured more tightly to each other. As shown in FIG. 12, a protrusion 20 may be provided at the proximal end of the tapered surface 16a, and a recess 21 to be engaged with the protrusion 20 may be provided at the proximal end of the tapered inner surface 15a. Consequently, the coil sheath 2 and the twisted wire 5 can be secured more tightly to each other at the position where the protrusion 20 is engaged with the recess 21.

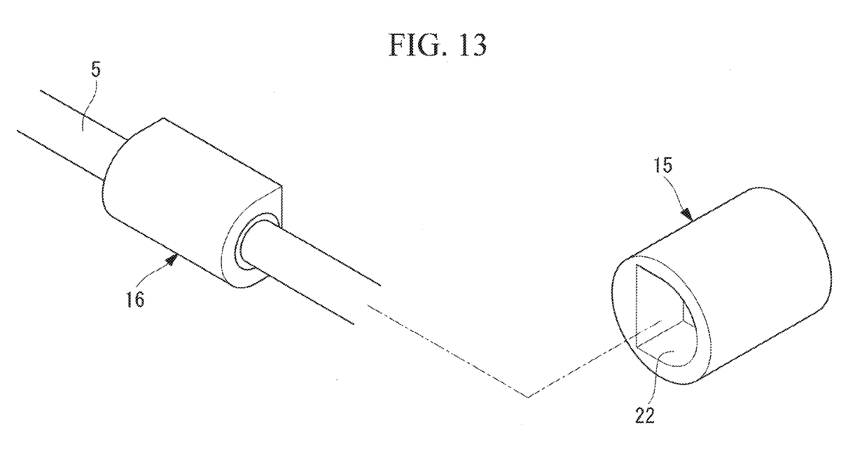

The protrusion 18 and the slit 19 are described above as a mechanism for attaching the first friction member 15 to the coil sheath 2 in a movable manner along the longitudinal axis and restricted rotation around the longitudinal axis. Alternatively, for example, as shown in FIG. 13, the outer surface of the first friction member 15 may have a noncircular cross-sectional shape, and the coil sheath 2 may have a hole 22 with a cross-sectional shape complementary to the cross-sectional shape of the first friction member 15.

As a result, the following aspect is read from the above described embodiment of the present invention.

An aspect of the present invention provides a surgical-device rotation mechanism including: a long member formed tubular shape; a rotation member that is rotatably supported by a distal end of the long member around a longitudinal axis of the long member and to which an end effector is fixed; a tension convertion member that is disposed inside the long member along the longitudinal axis of the long member, and has a distal end to which the rotation member is fixed, and converts tension applied by pulling of a proximal end into rotation around the longitudinal axis at the distal end; and a rotation restriction mechanism that is disposed between the long member and the tension convertion member and that increases or decreases friction between the rotation restriction mechanism and at least one of an inner surface of the long member and an outer surface of the tension convertion member by being operated from a proximal end of the long member so as to restrict or not restrict relative rotation between the long member and the tension convertion member around the longitudinal axis.

According to this aspect, when a pulling force is applied to the proximal end of the tension convertion member, the tension applied to the tension convertion member is converted into a force that rotates the distal end around the longitudinal axis, so that the rotation member fixed to the distal end of the tension convertion member is rotated around the longitudinal axis of the long member. Consequently, the end effector fixed to the rotation member is also rotated around the longitudinal axis.

In this case, at any rotational position of the rotation member, the rotation restriction mechanism is operated from the proximal end of the long member so that the friction between the rotation restriction mechanism and at least one of the inner surface of the long member and the outer surface of the tension convertion member is increased, whereby relative rotation between the long member and the tension convertion member around the longitudinal axis is restricted. Consequently, the rotation member can be maintained in a secured state at any rotational angle position relative to the long member. Furthermore, by operating the rotation restriction mechanism to reduce the friction between the rotation restriction mechanism and at least one of the inner surface of the long member and the outer surface of the tension convertion member, the secured state is released, so that the rotation member can be switched to a rotatable state.

In the above aspect, the rotation restriction mechanism may include a friction member fixed to one of the inner surface of the long member and the outer surface of the tension convertion member, and may also include an operation device that increases or decreases friction between the friction member and the other one of the inner surface of the long member and the outer surface of the tension convertion member.

Accordingly, in a case where the friction member is fixed to the inner surface of the long member, the friction between the friction member and the outer surface of the tension convertion member is increased or decreased by operating the operation device. In a case where the friction member is fixed to the outer surface of the tension convertion member, the friction between the friction member and the inner surface of the long member is increased or decreased by operating the operation device. This makes it possible to switch between a state where the rotation member is secured at any rotational angle position relative to the long member and a state where the secured state is released such that the rotation member is rotatable.

In the above aspect, the rotation restriction mechanism may include a friction member disposed in a gap between the inner surface of the long member and the outer surface of the tension convertion member, and may also include an operation device that increases or decreases friction between the friction member and the inner surface of the long member and friction between the friction member and the outer surface of the tension convertion member.

Accordingly, the friction between the friction member and the inner surface of the long member and the friction between the friction member and the outer surface of the tension convertion member are increased or decreased by operating the operation device. This makes it possible to switch between a state where the rotation member is secured at any rotational angle position relative to the long member and a state where the secured state is released such that the rotation member is rotatable.

In the above aspect, the friction member may be a balloon that is expandable or contractible in a radial direction, and the operation device may be a pipe capable of supplying a fluid to the balloon from the proximal end of the long member via a gap between the inner surface of the long member and the outer surface of the tension convertion member.

Accordingly, by supplying a fluid to the pipe at the proximal end of the long member, the balloon disposed between the inner surface of the long member and the outer surface of the tension convertion member is expanded, so that the friction between the balloon and the inner surface of the long member and the friction between the balloon and the outer surface of the tension convertion member are increased, whereby the rotation member can be maintained in a secured state at any rotational angle position relative to the long member. In contrast, by discharging the fluid from the balloon via the pipe, the balloon is contracted, so that the friction between the balloon and the inner surface of the long member and the friction between the balloon and the outer surface of the tension convertion member are decreased, whereby the rotation member becomes rotatable relative to the long member.

In the above aspect, the friction member may be an expansion-contraction member expandable or contractible in a radial direction by bending or flexing, and the operation device may be a power transmission member that transmits power to the expansion-contraction member from the proximal end of the long member via a gap between the inner surface of the long member and the outer surface of the tension convertion member.

Accordingly, by supplying power to the power transmission member at the proximal end of the long member, the expansion-contraction member disposed between the inner surface of the long member and the outer surface of the tension convertion member is expanded in the radial direction by being bent or flexed, so that the friction between the expansion-contraction member and the inner surface of the long member or the outer surface of the tension convertion member is increased, whereby the rotation member can be maintained in a secured state at any rotational angle position relative to the long member. In contrast, by stopping the supply of power to the power transmission member to cause the expansion-contraction member to contract in the radial direction, the friction between the expansion-contraction member and the inner surface of the long member or the outer surface of the tension convertion member is decreased, whereby the rotation member becomes rotatable relative to the long member.

In the above aspect, the rotation restriction mechanism may include a first friction member attached to the inner surface of the long member and that is configured to move along the longitudinal axis and restricted rotation around the longitudinal axis, a second friction member fixed to the outer surface of the tension convertion member, and a power transmission member that transmits power for moving the first friction member along the longitudinal axis. The second friction member may be provided with a tapered surface that gradually tapers toward a distal end. The first friction member may be provided with a tapered inner surface complementary to the tapered surface. Relative rotation between the long member and the tension convertion member around the longitudinal axis may be restricted by friction between the tapered surface and the tapered inner surface.

Accordingly, by supplying power to the power transmission member at the proximal end of the long member, the first friction member disposed between the inner surface of the long member and the outer surface of the tension convertion member is moved toward the proximal end, so that the first friction member is brought closer toward the second friction member, thereby bringing the two friction members into contact with each other. Consequently, the tapered surface provided in the second friction member comes into contact with the tapered inner surface provided in the first friction member, so that the rotation member can be maintained in a secured state at any rotational angle position relative to the long member in accordance with the friction between the two friction members. Because the friction between the tapered inner surface and the tapered surface is increased by bringing the two surfaces closer toward each other in the axial direction, the frictional force is readily increased, whereby relative rotation between the long member and the rotation member can be inhibited more reliably.

### REFERENCE SIGNS LIST

1 surgical-device rotation mechanism
2 coil sheath (long member)
3 end effector (surgical device)
4 rotation member
5 twisted wire (tension convertion member)
6, 11, 14 rotation restriction mechanism
8 balloon (friction member)
9 pipe (operation device)
12 plate spring member (expansion-contraction member, friction member)
13 push-fit coil (power transmission member, operation device)
15 first friction member
15a tapered inner surface
16 second friction member
16a tapered surface
17 wire (power transmission member)

1. A surgical-device rotation mechanism comprising:
a long member formed tubular shape;
a rotation member that is rotatably supported by a distal end of the long member around a longitudinal axis of the long member;
an end effector that is fixed to the rotating member;
a tension convertion member that is disposed inside the long member along the longitudinal axis of the long member and has a distal end to which the rotation member is fixed; and
a rotation restriction mechanism that is disposed between the long member and the tension convertion member,
wherein the tension convertion member is configured to convert tension applied by pulling of a proximal end into rotation around the longitudinal axis at the distal end, and
wherein the rotation restriction mechanism is configured to increase or decrease friction between the rotation restriction mechanism and at least one of an inner surface of the long member and an outer surface of the tension convertion member by being operated from a proximal end of the long member so as to restrict or not restrict relative rotation between the long member and the tension convertion member around the longitudinal axis.

2. The surgical-device rotation mechanism according to claim 1,
wherein the rotation restriction mechanism further comprises:
a friction member fixed to one of the inner surface of the long member and the outer surface of the tension convertion member; and
an operation device that is configured to increase or decrease friction between the friction member and the other one of the inner surface of the long member and the outer surface of the tension convertion member.

3. The surgical-device rotation mechanism according to claim 1,
wherein the rotation restriction mechanism further comprises:
a friction member disposed in a gap between the inner surface of the long member and the outer surface of the tension converting member; and
an operation device that is configured to increase or decrease friction between the friction member and the inner surface of the long member and friction between the friction member and the outer surface of the tension convertion member.

4. The surgical-device rotation mechanism according to claim 2,
wherein the friction member comprises a balloon that is configured to expand or contract in a radial direction, and
wherein the operation device comprises a pipe that is configured to supply a fluid to the balloon from the proximal end of the long member via a gap between the inner surface of the long member and the outer surface of the tension convertion member.

5. The surgical-device rotation mechanism according to claim 3,
wherein the friction member comprises a balloon that is configured to expand or contract in a radial direction, and
wherein the operation device comprises a pipe that is configured to supply a fluid to the balloon from the proximal end of the long member via a gap between the inner surface of the long member and the outer surface of the tension convertion member.

6. The surgical-device rotation mechanism according to claim 2,
wherein the friction member comprises an expansion-contraction member that is configured to expand or contract in a radial direction by bending or flexing, and
wherein the operation device comprises a power transmission member that is configured to transmit power to the expansion-contraction member from the proximal end of the long member via a gap between the inner surface of the long member and the outer surface of the tension convertion member.

7. The surgical-device rotation mechanism according to claim 3,
wherein the friction member comprises an expansion-contraction member that is configured to expand or contract in a radial direction by bending or flexing, and
wherein the operation device comprises a power transmission member that is configured to transmit power to the expansion-contraction member from the proximal end of the long member via a gap between the inner surface of the long member and the outer surface of the tension convertion member.

8. The surgical-device rotation mechanism according to claim 1,
wherein the rotation restriction mechanism further comprises:
a first friction member that is disposed to the inner surface of the long member and that is configured to move along the longitudinal axis and be restricted rotation around the longitudinal axis;
a second friction member that is fixed to the outer surface of the tension convertion member; and
a power transmission member that is configured to transmit power for moving the first friction member along the longitudinal axis,
wherein the second friction member is provided with a tapered surface that gradually tapers toward a distal end,
wherein the first friction member is provided with a tapered inner surface complementary to the tapered surface, and
wherein relative rotation between the long member and the tension converting member around the longitudinal axis is restricted by friction between the tapered surface and the tapered inner surface.
